(19) United States
(12) Patent Application Publication
(10) Pub. No.: US 2005/0176003 A1
(43) Pub. Date: Aug. 11, 2005

(54) Plastic substrate for microchips

(75) Inventors: Kaneshisa Yokoyama, Akita-shi (JP); Hiroshi Sawai, Akita-Shi (JP); Hideyuki Shimaoka, Akita-Shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: SUMITOMO BAKELITE CO., LTD., Tokyo (JP)

(21) Appl. No.: 10/495,743

(22) PCT Filed: Nov. 15, 2002
(86) PCT No.: PCT/JP02/11938

(30) Foreign Application Priority Data
Nov. 27, 2001 (JP) 2001-361047
Dec. 17, 2001 (JP) 2001-382448
Mar. 14, 2002 (JP) 2002-70812

(51) Int. Cl. C12M 3/00

(52) U.S. Cl. 435/6; 435/287.2

(57) ABSTRACT

This invention relates to a plastic substrate for a microarray chip, which is characterized in that an aminoalkylsilane with an aldehyde group derived from glutaraldehyde and introduced onto an amino group of the aminoalkylsilane exists on a surface of the plastic substrate, and also to a process for its production and a method of its use. Substrates according to the present invention have no variations in the immobilized amount of DNA fragments upon immobilization of DNA fragments, permit reproducible hybridization with high efficiency, and are excellent in storability.

### TECHNICAL FIELD

This invention relates to a plastic substrate for a microarray chip, said plastic substrate being high in the immobilization efficiency and hybridization efficiency of DNA fragments, and also to a process for its production and a method of its use.

### BACKGROUND ART

Methods of immobilizing single strands of DNA fragments onto a substrate can be divided roughly into methods relying upon adsorption and methods involving the formation of covalent bonds.

The immobilization of DNA fragments onto a substrate by adsorption makes use of the negative charges of DNA. Positive charges are applied to a surface of the substrate, and by electric attraction, DNA fragments are adsorbed and immobilized.

As a substrate for use in such a method, one coated on a surface thereof with polylysine is employed. In general, however, polylysine has poor stability after coating and can hardly be stored over a long time. Subsequent to the coating of a substrate with polylysine, its immobilization ability for DNA fragments declines with time. Moreover, the storability of the substrate after immobilization of DNA thereon is also poor, and in storage at room temperature, the substrate can be stored for only two weeks or so.

As another method for applying positive charges likewise, there is a method that comprises reacting an amino-containing silane coupling agent with a glass substrate to introduce amino groups.

Glass is used in conventional substrates, so that silane coupling agents having good compatibility with glass are used. The reaction of a silane coupling agent with a glass substrate is easy, so that use of an amino-containing silane coupling agent facilitates introduction of amino groups onto a surface of the glass substrate.

For the immobilization of long DNA strands composed of several hundreds to several thousands of bases, it is effective to introduce amino groups and to have the DNA strands adsorbed by making use of the negative charges of the DNA strands. The introduction of amino groups by an aminosilane coupling agent can bring about high storage stability, and is an effective method as a substitute for the above-described polylysine coating.

It is, however, difficult to apply this method to the immobilization of short DNA strands composed of as few as several tens of bases or so, that is, so-called oligo DNA.

In the case of short DNA strands, no stable immobilization of the DNA is feasible insofar as adsorption alone is merely relied upon, because separation of the DNA takes place after its spotting onto a substrate and further, in the detection of the DNA by hybridization, the hybridization does not take place efficiently so that the detection of the DNA is impossible or variations occur in the detection intensity.

With a view to overcoming the above-mentioned problems, methods have then been proposed to form covalent bonds such that DNA is immobilized on a surface of a substrate. The best known method comprises using an immobilizing substrate with amino groups introduced by a silane coupling agent as described above, introducing, on the other hand, amino groups to ends of DNA fragments, and then immobilizing the DNA fragments to a surface of the substrate via covalent bonds while using a crosslinking agent such as glutaraldehyde.

In the case of a glass substrate, it carries a number of hydroxyl groups on its surface so that an amino-containing silane coupling agent can be introduced at high density. The introduction of amino groups at high density may imply to result in many bonding sites, thereby presumably making it possible to efficiently immobilize DNA fragments. When amino groups derived from the silane coupling agent are carried at high density, however, the formation of covalent bonds by crosslinking with glutaraldehyde as described above means that the addition of glutaraldehyde results in crosslinking of amino groups themselves of the silane coupling agent and inhibits the formation of covalent bonds with the amino groups on the side of the DNA strands to result in a reduced immobilized amount of DNA fragments.

It may be contemplated to lower the concentration of the silane coupling agent such that the density of amino groups is reduced adequately. When hydroxyl groups remain in the free form without any reaction with the silane coupling agent, however, the overall charge of the surface is negative. It is, therefore, difficult for the negatively-charged DNA strands to come close to the surface. As a result, the DNA strands are prevented from bonding to the aldehyde groups introduced to the ends of the amino groups, thereby making it difficult to immobilize DNA fragments in any sufficient amount.

For the reasons mentioned above, it has been difficult to obtain, from a glass substrate, a substrate which is highly efficient in DNA immobilization and hybridization, has high reproducibility, and is suitable for a DNA microarray of the Stanford University type.

Objects of the present invention are to provide a DNA immobilizing substrate which, upon employing it as a substrate for use in the production of a DNA microarray, has a high immobilization efficiency for DNA fragments, is even in the immobilized amount of DNA fragments throughout the substrate, and has high hybridization efficiency and high reproducibility in the hybridization between the immobilized DNA fragments and their target DNA strands; and also to provide a DNA-immobilizing substrate having high quality storability such that the immobilization and hybridization of DNA fragments can be stably performed even after the production of the substrate.

### DISCLOSURE OF THE INVENTION

To solve such conventional problems as described above, the present inventors have proceeded with an extensive investigation. As a result, it has been found that, when a hydrophilization treatment is applied to a surface of a plastic substrate, amino groups are introduced with an aminoalkylsilane and aldehyde groups are then introduced into the amino groups with glutaraldehyde, a high immobilization efficiency of DNA fragments can be achieved with good reproducibility and even after the introduction of the aldehyde groups, the quality of the plastic substrate remains stable and the reactivity of the aldehyde groups is retained. These findings have led to the completion of the present invention.

Specifically, the present invention provides a plastic substrate for a microarray chip, characterized in that an aminoalkylsilane with an aldehyde group derived from glutaraldehyde and introduced in an amino group of said aminoalkylsilane exists on a surface of the plastic substrate.

The present invention also provides a process for the production of a plastic substrate for a microarray chip, characterized in that an aminoalkylsilane with an aldehyde group derived from glutaraldehyde and introduced in an amino group of said aminoalkylsilane is caused to exist on a surface of the plastic substrate by steps which comprise:
(1) subjecting a starting plastic substrate to a surface oxidation treatment,
(2) bringing the thus-obtained plastic substrate into contact with a solution of the aminoalkylsilane, and
(3) bringing the resulting plastic substrate into contact with a solution of glutaraldehyde.

The present invention further provides a method of use of a plastic substrate for a microarray chip characterized in that the method comprises dissolving in a solution DNA strands with amino groups introduced at ends thereof, bringing the resulting solution into contact with the above-described plastic substrate, and having the amino groups of the DNA strands and aldehyde groups, which have been introduced onto the substrate, covalently bonded with each other to immobilize the DNA strands on the substrate.

### BRIEF DESCRIPTION OF THE DRAWINGS

FIG. 1 is a schematic illustration showing an embodiment of the shape of a substrate according to the present invention.

FIG. 2 is a schematic illustration showing another embodiment of the shape of the substrate according to the present invention.

FIG. 3 is a schematic illustration depicting a further embodiment of the shape of the substrate according to the present invention.

FIG. 4 is a schematic illustration depicting a still further embodiment of the shape of the substrate according to the present invention.

FIG. 5 histogrammatically illustrates distributions of intensity of background fluorescence in Referential Example 4 and Comparative Referential Example 5.

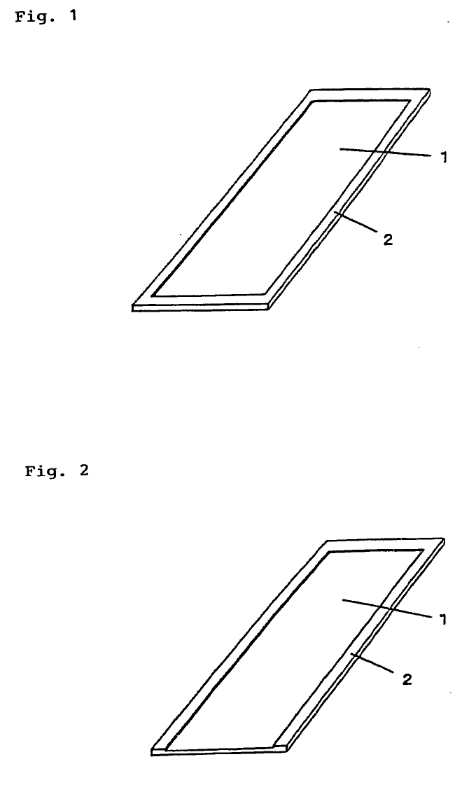

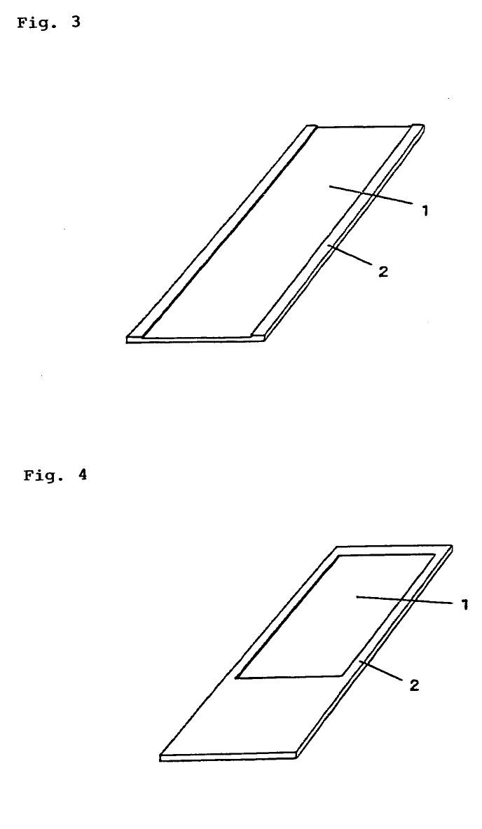

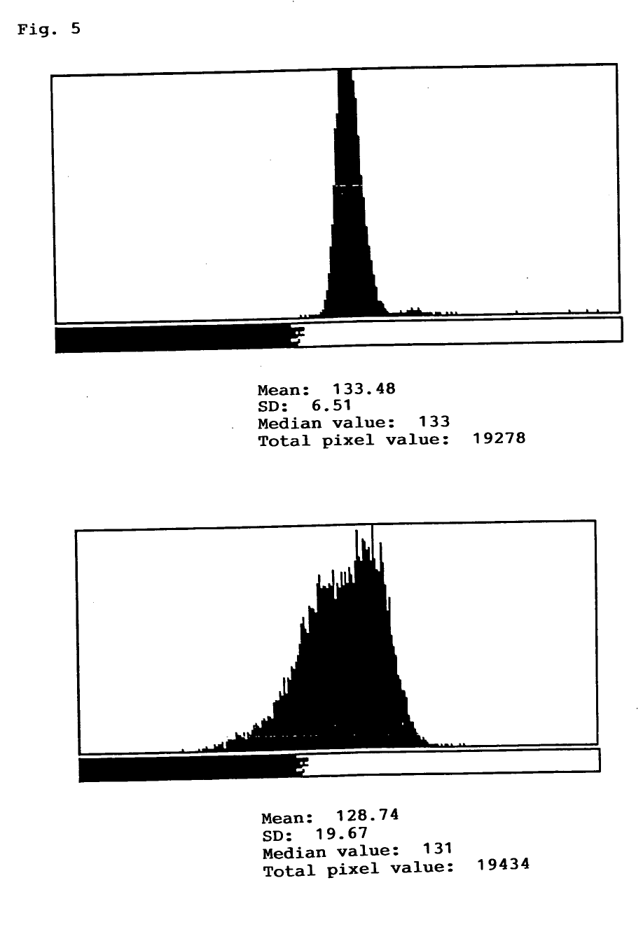

In each of FIGS. 1 through 4, numeral 1 designates a back side of a sample-immobilizing section, and numeral 2 indicates a thickened portion.

### BEST MODES FOR CARRYING OUT THE INVENTION

The plastic substrate for the microarray chip, according to the present invention, is characterized in that an aminoalkylsilane with an aldehyde group derived from glutaraldehyde and introduced in an amino group of the aminoalkylsilane exists on a surface of the plastic substrate. Preferred as the aminoalkylsilane is an aminoalkyltrialkoxysilane, for example, a compound represented by the following formula (1): 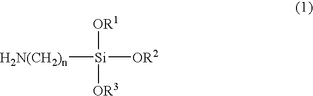
wherein n stands for an integer of from 1 to 16, and R¹, R² and R³ represent alkyl groups, respectively. In the formula (1), n may preferably be from 1 to 10, with 1 to 8 being particularly preferred. As the alkyl groups represented by R¹, R² and R³, alkyl groups having 1 to 6 carbon atoms are preferred, with alkyl groups having 1 to 4 carbon atoms such as methyl, ethyl and propyl groups being particularly preferred.

Glutaraldehyde (OHC(CH₂)₃CHO) has been introduced in the amino group of the aminoalkylsilane. Specifically, the aldehyde and amino group form a Schiff base (—CH═N—) or its reduction derivative (—CH₂NH—).

On the surface of the substrate according to the present invention, an aminoalkylsilane with no aldehyde group introduced therein may exist in combination with the above-described aldehyde-introduced aminoalkylsilane. As the aminoalkylsilane with no aldehyde group introduced therein, one represented by the formula (1) can be mentioned.

In addition, an alkylsilane may further exists in combination on the surface of the substrate according to the present invention. Preferred examples of the alkylsilane include compounds represented by the following formula (2): 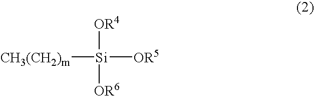
wherein m stands for an integer of from 1 to 16, and R⁴, R⁵ and R⁶ represent alkyl groups, respectively. In the formula (2), m may preferably be from 1 to 8, with 1 to 6 being particularly preferred. As the alkyl groups represented by R⁴, R⁵ and R⁶, alkyl groups having 1 to 6 carbon atoms are preferred, with alkyl groups having 1 to 4 carbon atoms such as methyl, ethyl and propyl groups being particularly preferred.

Observing the surface of the substrate according to the present invention at the molecular level, molecular chains having aldehyde groups at free ends thereof and molecular chains having no reactive groups (aminoalkylsilane and/or alkylsilane) are considered to exist in such a form as extending upright close together. When the aminoalkylsilane and the alkylsilane are caused to exist in combination, an adjustment to their proportions makes it possible to maintain a sufficient distance between amino groups themselves such that the crosslinking of the amino groups themselves with glutaraldehyde can be prevented to assure the efficient introduction of aldehyde groups.

Upon mixing the aminoalkylsilane and the alkylsilane with each other, it is preferred to mix the alkylsilane in a range of from 20 to 400 parts by weight per 100 parts by weight of the aminoalkylsilane although their mixing ratio varies depending on the number of hydroxyl groups introduced onto the substrate. An amount of less than 20 parts by weight results in the incapability of recognizing the effect which would otherwise be available from the combined existence of the alkylsilane, while an amount of greater than 400 parts by weight leads to a reduction in the density of aldehyde groups so that the immobilized amount of DNA per unit area decreases.

Depending on the length of DNA to be hybridized finally, the mixing proportion of the alkylsilane may be adjusted. For raising the efficiency of hybridization, it is effective to increase the mixing proportion of the alkylsilane when the length of DNA is long but to decrease the mixing proportion of the alkylsilane when the length of DNA is short.

The molecular chains of the aminoalkylsilane and alkylsilane may preferably be linear, and bent molecular chains due to the inclusion of nitrogen atoms or the like at intermediate points thereof are not preferred. Use of those having bent molecular chains makes it impossible to form the state that, as mentioned above, molecular chains extend upright close together while the molecular chains are supporting each other, so that the efficiency of DNA immobilization is considerably lowered.

Further, use of an alkylsilane having a shorter molecular chain length than the associated aminoalkylsilane allows aldehyde groups and DNA fragments, in which amino groups have been introduced, to react efficiently so that a high DNA immobilization rate can be obtained, because the molecular chains with aldehyde groups contained therein assume the form that the molecular chain portions having the aldehyde groups therein lie in a top layer while being supported by the molecular chains of the alkylsilane. In addition, the DNA strands so immobilized are fixed with a distance kept from the surface of the substrate so that in the subsequent hybridization step, the DNA fragments are facilitated to undergo homologization and the efficiency of hybridization can be increased.

The substrate according to the present invention is a plastic substrate. No particular limitation is imposed on the plastic for use in the substrate insofar as it is equipped with good moldability, waterproofness and heat resistance. However, resins which do not give off much fluorescence are preferred because of the extensive use of DNA detection methods involving the modification of DNA with a fluorescent label. Examples of such resins include saturated cyclic polyolefin resins and fluoroplastics.

Of these, saturated cyclic polyolefin resins are suited, because they have high heat resistance and are low in autofluorescence in the wavelength range of Cy3 and Cy5, fluorochromes widely used in DNA microarrays. Fluoroplastics are low in autofluorescence over a wide wavelength range, but are accompanied with the drawbacks that they require removal of fluorine produced during molding and their molding is difficult.

Preferred as the saturated cyclic polyolefin resins are saturated polymers obtained by hydrogenating polymers having a cyclic olefin structure or copolymers between cyclic olefins and α-olefins.

Examples of the former resins include hydrogenation products of ring-opening polymerization products of norbornene, which are represented by the following formula (3): 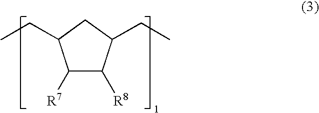
wherein R⁷ and R⁸ may be the same or different and each represents a hydrogen or a hydrocarbon residual group having 1 to 10 carbon atoms, or R⁷ and R⁸ may be fused together to form a ring.

The polymers having structural units represented by the formula (3) are saturated polymers, which are produced by using, as monomers, norbornene and its alkyl- or alkylidene-substituted derivatives, specifically 5-methyl-2-norbornene, 5,6-dimethyl-2-norbornene, 5-ethylidene-2-norbornene and the like and further, dicyclopentadiene, 2,3-dihydrodicyclopentadiene and their alkyl derivatives such as their methyl and ethyl derivatives, subjecting these monomers to ring-opening polymerization, and then hydrogenating the resultant ring-opening polymerization products.

It is also possible to use saturated polymers, which are produced by hydrogenating polymers of cyclic olefin monomers represented by the formula (4) or random copolymers between α-olefins such as ethylene, propylene, isopropylene, 1-butene, 3-methyl-1-butene, 1-pentene and 1-hexene and the cyclic olefin monomers represented by the formula (4). 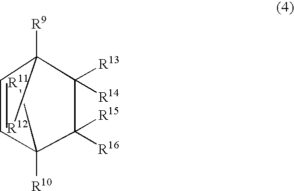
wherein R⁹ to R¹⁶ are each selected from the group consisting of hydrogen, halogen atoms and hydrocarbon residual groups, or R¹³ and R¹⁶ may be fused together to form a ring.

Further, it is also possible to use saturated polymers, which are produced by hydrogenating polymers of cyclic olefin monomers represented by the formula (5) or random copolymers between α-olefins such as ethylene, propylene, isopropylene, 1-butene, 3-methyl-1-butene, 1-pentene and 1-hexene and the cyclic olefin monomers represented by the formula (5). 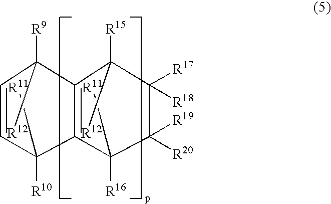
wherein R⁹ to R²⁰ are each selected from the group consisting of hydrogen, halogen atoms and hydrocarbon residual groups, or R¹⁷ to R²⁰ may be fused together to form a ring, and p stands for a number of 1 or greater.

Among these, particularly preferred are saturated cyclic polyolefins in each of which at least one of norbornene and norbornene derivatives (for example, compounds of the formula (3)) is contained as monomer units.

No particular limitation is imposed on the form of the substrate according to the present invention insofar as it can be used to immobilize and measure an organism-derived substance. Examples include plates as well as beads and the like for immunological analysis, which are widely employed in the field of immunological analyses. In particular, plate-like forms useful as DNA microarrays are most suited.

The substrate according to the present invention is also useful in a method that a sample, which has been subjected to a treatment to add a fluorescent label to the sample, is detected based on fluorescence given off by feeding excitation light. In such a case, a pigment can preferably be included in a plastic for use in the substrate to make the plastic opaque such that penetration of the excitation light into the substrate can be prevented and fluorescence, which would otherwise be given off by the excitation light, is no longer allowed to reach detectors.

The pigment can be either a black pigment or a white pigment. However, a black pigment is preferred, with carbon black being more preferred for its coloring power, heat resistance, bleed resistance and chemical resistance.

To detect on a microarray chip a fluorescent label which has been added to an extremely trace amount of a sample, there is a method which is often employed. According to this method, the detection is performed by irradiating strong laser light as excitation light to increase the intensity of fluorescence. If the content of a pigment added in the plastic is smaller than 1 wt. %, the opacity is insufficient so that laser light is not blocked but is allowed to penetrate into the plastic. As a consequence, background fluorescence noise produced from the plastic leaks out, and is detected as a value which is sufficiently large relative to a signal from the florescent label. At least 1 wt. % of a pigment is, therefore, needed to prevent laser light from penetrating into the substrate from its surface and also, to prevent fluorescence, which is produced in a surface molecular layer of the substrate, from leaking out of the surface molecular layer. By increasing the content of the pigment, the intensity of fluorescence which leaks out of the surface molecular layer is decreased, thereby making it possible to decrease the intensity of background fluorescence.

If the pigment is added at a content higher than 60 wt. %, on the other hand, the content of the plastic in the substrate is lowered so that the inherent moldability, hardness, heat resistance, wettability, sample adsorbability and other physical or chemical properties of the plastic vary substantially to possibly cause problems in use. Accordingly, such a high content is not suited either. Insofar as a pigment is added at a content not high than 60 wt. % in a resin, the physical or chemical properties of the resin are expected to undergo no substantial changes. Needless to say, the lower the content of the pigment, the smaller the changes in physical or chemical properties as compared with the inherent ones of the resin, and the more preferred in conducting the designing of a microarray chip.

With the foregoing in view, it is possible to reduce the intensity of fluorescence, which is given off from the plastic of the substrate, and to clearly detect a signal from the fluorescent label without practically changing the physical or chemical properties of the plastic by controlling the content of the pigment to 1 to 60 wt. %, preferably 1.5 to 20 wt. %, more preferably 2 to 5 wt. % in the plastic.

To avoid deformation of a sample-immobilizing section of the substrate according to the present invention by the application of uneven force from a substrate-fixing jig of a measuring system, the substrate according to the present invention may preferably be provided on the back side thereof with a means for limiting the area of contact with the measuring system. As the means for limiting the area of contact, it is preferred to arrange a raised portion at a part of the back side of the sample-immobilizing section. It is more preferred to arrange the raised portion in the form of an outer edge portion greater in thickness than the sample-immobilizing section.

A more detailed description will be made about the sample-immobilizing section of the substrate according to the present invention. When an evaluation is performed using a confocal laser scanner, occurrence of deformation such as a strain or warp at the sample-immobilizing section of a DNA microarray substrate makes it difficult to effect focusing so that no accurate data are available. As a cause of the deformation of the substrate, force which the substrate receives from a substrate-fixing jig in the scanner is responsible in many instances. To avoid deformation under the force from the jig, certain countermeasures can be mentioned including (A) to increase the stiffness of the substrate and (B) to form the substrate into such a structure that the sample-immobilizing section and the jig do not come into direct contact with each other to minimize the transmission of force to the sample-immobilizing section.

Specific examples of the countermeasure (A) include inter alia to increase the thickness of the substrate and to increase the strength of the material itself by incorporation of a filler. However, an increase in the thickness of the substrate involves a serious potential problem that conventional equipment and tools may become no longer usable. Further, the countermeasure relying upon the incorporation of a filler is accompanied by problems such as accelerated abrasion of a molding die and changes in surface treatment properties, and is not preferred from the practical standpoint. On the other hand, the countermeasure (B) does not develop such problems as mentioned above. Namely, the thickness of the substrate as a whole is not different from the conventional substrate so that no replacement is needed for the equipment and tools. Further, the materials themselves of the substrate are not changed so that no problem arises in manufacture.

The construction of the substrate-immobilizing jig of the scanner considerably varies depending on the model. The construction which is used most commonly these days is of the type that the lower side of a substrate is pressed upwardly by a leaf spring against an upper jig to hold the substrate in place. According to this type, the substrate may be pressed upward at a central section thereof to develop a strain in the measuring surface if the pressing force of the leaf spring is too large. The application of an uneven stress to the sample-immobilizing section can be eliminated to avoid deformation by arranging a region, which protrudes beyond the sample-immobilizing section, on an outer edge portion of the substrate such that the contact with the jig is limited only to the raised portion. As the shape of the raised portion, it is preferred, as shown in FIGS. 1 through 4, to arrange a region resembling the frame of a picture frame at and along an outer edge portion of the back side of the sample-immobilizing section and to make the thickness of the region greater than the sample-immobilizing section. By making the outer edge portion thicker, the stiffness of this region increases to bear a large majority of force from the substrate-fixing jig. A stress applied to the sample-immobilizing section is, therefore, reduced so that deformation can be avoided.

As the deformation by the force from the substrate-fixing jig is 20 to 100 μm or so, the difference in thickness between the sample-immobilizing section and the outer edge portion may preferably be from 20 to 500 μm, more preferably from 20 to 200 μm, most preferably from 20 to 100 μm.

The formation of the raised portion into the above-described shape can bring about the advantageous effect that the sample-immobilizing section is protected from scratches or like damage upon handling the substrate. Described specifically, in the case of a substrate both sides of which are planar in general, minute scratches or the like are formed on the sample-immobilizing side or its back side when the substrate is placed on a working bench or the like. Upon measurement, these minute scratches or the like may be detected as noise. In the case of the substrate according to the present invention, however, such direct contact does not take place so that the possibility of scratching or otherwise damaging the substrate is reduced and a high-accuracy measurement is feasible.

A detailed description will hereinafter be made about the plastic substrate for the microarray chip, the process for its production and the method of its use, all of which pertain to the present invention.

The substrate according to the present invention is produced by causing an aminoalkylsilane, in which an aldehyde group derived from glutaraldehyde has been introduced in an amino group of the aminoalkylsilane, to exist on a surface of the plastic substrate by steps which comprise:
(1) subjecting a starting plastic substrate to a surface oxidation treatment,
(2) bringing the thus-obtained plastic substrate into contact with a solution of said aminoalkylsilane, and
(3) bringing the resulting plastic substrate into contact with a solution of glutaraldehyde.

The oxidation treatment of the surface of the substrate is a step to introduce hydroxyl groups onto the surface of the substrate. Illustrative oxidation treatments include low-temperature plasma treatment, corona discharge treatment, flame treatment, and other chemical treatments. As a method that permits a stable and uniform oxidation treatment to a resin surface, a treatment by low-temperature plasma is preferred. As a gas species for use in low-temperature plasma treatment, use of oxygen is preferred because stable introduction of hydroxyl groups is feasible. Accordingly, it is preferred to conduct low-temperature plasma treatment under oxygen for oxidation treatment or an oxygen-containing gaseous atmosphere.

Immediately after the introduction of hydroxyl groups onto the surface of the substrate in the above-described manner, hydroxyl groups may preferably be introduced further onto carbon atoms which are in a radical state or are π-bonded. As a method for introducing hydroxyl groups at this stage, a method that can provide an opportunity of contacting with water molecules in the treatment or a post-treatment is preferred. It may be contemplated to immerse the substrate in a solution such as a dilute alkaline aqueous solution of a permanganate salt, a mixed alcohol-water solvent or pure water, to immerse the substrate in concentrated sulfuric acid and then in pure water, or to bring the substrate into contact with an atmosphere the humidity of which is from 80 to 100%. Among these methods, the immersion in pure water is most suited because it is simple and convenient, is applicable without any limitation to the substrate shape and is free of any troublesome waste treatment.

When oxygen-gas low-temperature plasma discharge treatment is conducted as the oxidation treatment, the carbon atoms on the surface of the substrate are expected to be converted into a radical state or n-bonded state by oxygen radicals. With polystyrene or polycarbonate which is another kind of resin having high light transmittance, aromatic rings are inherently contained in the molecular structure of the polymer, so that fluorescence from the resin itself is strong and fluorescence increased by the oxidation treatment does not cause a problem as noise.

However, π-bonds are not contained in the molecular structure of a saturated cyclic polyolefin resin such as a norbornene resin, and the inherent intensity of fluorescence of the resin itself is very small. When oxygen-gas low-temperature plasma discharge treatment is applied, about 10 to 25% of the carbon atoms which exist on the surface of the substrate have π-bonds. This is considered to have served as a cause of the increased fluorescence from the substrate itself in the oxygen-gas low-temperature plasma discharge treatment.

Concerning the increased fluorescence from the substrate itself by the oxygen-gas low-temperature plasma discharge treatment, it is possible to introduce hydroxyl groups onto carbon atoms in a radical state and to reduce the increase in the autofluorescence as a result of the oxidation treatment even by simply immersing the substrate in pure water immediately after the oxygen-gas low-temperature plasma discharge treatment. When the percentage of carbon atoms having π-bonds is 15% or less in the carbon atoms existing in the molecular layer which forms the surface of the substrate, noise of autofluorescence does not interfere with the measurement. Therefore, the percentage of carbon atoms having π-bonds in the carbon atoms, which exist in the molecular layer forming the surface of the substrate, may preferably be 15% or less, with 10% or less being more preferred.

By applying the above-described treatment, many hydroxyl groups can be introduced onto the surface of the substrate, thereby bringing about the advantageous effect that more reaction sites are available upon application of the surface treatment to the substrate. When an aminosilanating agent is coated as disclosed, for example, in JP 60-15560 A, the aminosilanating agent can be coated in a greater amount as more hydroxyl groups exist on the surface of the substrate. Compared with the application of only oxygen-gas low-temperature plasma discharge treatment to the substrate of the saturated cyclic polyolefin resin, the substrate immersed in pure water immediately after oxygen-gas low-temperature plasma discharge treatment contains hydroxyl groups as many as about 1.5 times in the molecular layer which forms the surface of the substrate.

The substrate is next brought into contact with the aminoalkylsilane such that the hydroxyl groups introduced onto the surface of the substrate and the aminoalkylsilane are reacted to introduce amino groups. The introduction of the amino groups onto the surface of the substrate is conducted by preparing a solution with the aminoalkylsilane dissolved in an organic solvent such as methanol, immersing into the solution the substrate the surface of which has been subjected to the oxidation treatment, and then allowing the substrate to stand in the solution. After the reaction, the substrate is taken out of the solution and is then washed. If a mixture of the aminoalkylsilane and the alkylsilane is used instead of the aminoalkylsilane in the above reaction, the aminoalkylsilane and the alkylsilane are introduced in combination onto the surface of the substrate.

Aldehyde groups are next introduced into the amino groups. Glutaraldehyde is dissolved into a solution, in which the substrate with the amino groups introduced thereon is immersed and left over. One of the aldehyde groups of glutaraldehyde is reacted with the amino group and, after the substrate is allowed to stand, the substrate is washed with ultrapure water and then dried to finally obtain the substrate with aldehyde groups introduced thereon. When the aminoalkylsilane and the alkylsilane exist in combination on the substrate, the plastic substrate is finally obtained with the aminoalkylsilane, which contains an aldehyde group derived from glutaraldehyde and introduced in the amino group thereof, and the alkylsilane existing in combination on the surface thereof.

A description will next be made about a method for immobilizing DNA by using the present invention.

As DNA to be immobilized on the surface of the substrate, oligo DNA formed of several tens of base chains is suited. Upon immobilization, amino groups are introduced onto the ends of DNA strands.

The DNA strands with the amino groups introduced thereon are dissolved in a DNA-immobilizing solution, spotted onto the substrate by a machine called “spotter”, and then allowed to stand for immobilization.

Conventionally, it is necessary to add glutaraldehyde in a solution beforehand upon immobilization of DNA strands on a substrate on which amino groups have been introduced. In the case of the substrate according to the present invention, however, glutaraldehyde has already been introduced so that no addition of glutaraldehyde to the solution is needed.

When glutaraldehyde is added to a DNA solution, amino groups introduced on the ends of the DNA strands undergo crosslinking by themselves, leading to a decrease in the number of DNA strands which can be actually immobilized. As no glutaraldehyde is added to the DNA solution, it is possible to reduce a loss to DNA fragments to be immobilized.

When aldehyde groups are introduced by the process of the present invention, the use of a saturated cyclic polyolefin resin as a resin for forming the substrate results in the introduction of many aldehyde groups, so that the immobilization rate of DNA strands formed of several tens to about 50 bases and called “oligo DNA” is high and the detection efficiency is high in the hybridization of DNA as a detection target.

To improve the detection efficiency by the hybridization of DNA, an appropriate distance is needed between immobilized DNA strands because DNA to be detected is in the form of long DNA strands having a base number of from 500 to 1,000 or so. In the case of a plastic surface, especially a surface of a saturated cyclic polyolefin resin, a distance most suited for conducting hybridization is considered to be maintained.

It has also been found that the DNA immobilization ability of the substrate is not recognized to drop even when the substrate is stored over a long time after the introduction of aldehyde groups. As a reason for this advantageous effect, the introduced amino groups seems to be controlled at an appropriate density so that crosslinking of the amino groups themselves would hardly take place.

### EXAMPLES

The present invention will hereinafter be specifically described based on Examples.

### Example 1

Using a saturated cyclic polyolefin resin (a hydrogenation product of a random copolymer between ethylene and dicyclopentadiene which is a norbornene derivative), slide-glass-shaped substrates were obtained by injection molding. To the surfaces of those molded products, a hydrophilization treatment was applied by low-temperature oxygen plasma treatment. Next, a solution with γ-aminopropytriethoxysilane dissolved as an aminoalkylsilane at 5% concentration in methanol was prepared as a treatment solution for the introduction of amino groups. After the molded products were immersed for 2 hours in the solution, the resulting substrates were taken out of the solution, allowed to stand in ultrapure water, taken out of the ultrapure water, and then dried. Glutaraldehyde was dissolved at 2% concentration in PBS(−) to prepare a glutaraldehyde solution. The substrates which had been subjected to the aminoalkylsilane treatment were immersed in the glutaraldehyde solution. After the substrates were allowed to stand there for 4 hours, they were taken out of the solution, immersed and washed in ultrapure water, and then dried.

### Example 2

The substrates produced in Example 1 were placed in cases for slide glasses. The cases with the substrates placed therein were put in laminated pouches of aluminum and PET. The pouches were sealed, and then, the substrates were stored for 6 months at room temperature.

### Comparative Example 1

Glass-made slide glasses were provided. Using γ-aminopropytriethoxysilane as an aminosilane coupling agent, it was dissolved at 5% concentration in methanol to prepare a treatment solution for the introduction of amino groups. After the slide glasses were immersed for 2 hours in the solution, the resulting substrates were taken out of the solution, immersed in ultrapure water, allowed to stand there, taken out of the ultrapure water, and then dried. Glutaraldehyde was dissolved at 2% concentration in PBS(−) to prepare a glutaraldehyde solution. The substrates which had been subjected to the aminoalkylsilane treatment were immersed in the glutaraldehyde solution. After the substrates were allowed to stand there for 4 hours, they were taken out of the solution, immersed and washed in ultrapure water, and then dried.

### Comparative Example 2

The substrates produced in Comparative Example 1 were placed in cases for slide glasses. The cases with the substrates placed therein were put in laminated pouches of aluminum and PET. The pouches were sealed, and then, the substrates were stored for 6 months at room temperature.

(Aminated Oligo DNA)

Synthesized was oligo DNA (hereinafter called “aminated oligo DNA”) with an amino group introduced to the 5′ end of oligo DNA which consisted of a sequence of

| | |
|---|---|
| 5′ -TAGAAGCATTTGCGGTGGACGATG-3′ | (SEQ ID NO: 1) |
| (Rhodamine-labeled oligo DNA). | |

Synthesized was oligo DNA (hereinafter called “rhodamine-labeled oligo DNA”) with rhodamine labeled on the 5′ end of oligo DNA which consisted of a sequence of

| | |
|---|---|
| 5′ -CATCGTCCACCGCAAATGCTTCTA-3′ | (SEQ ID NO: 2) |
| (Cy3-labeled cDNA). | |

Synthesized was a primer which consisted of the following sequences:

| | |
|---|---|
| Sense | |
| 5′ -TGACGGGGTCACCCACACTGTGCC-3′ | (SEQ ID NO: 3) |
| Antisense | |
| 5′ -TAGAAGCATTTGCGGTGGACGATG-3′ | (SEQ ID NO: 1) |

On the other hand, cDNA was provided from HeLa cells. Using the cDNA and the above-described primer, cDNA (hereinafter called “Cy3-labeled cDNA”) labeled with Cy3 and corresponding to β-actin of 661 bases was synthesized by PCR.
(Comparison in DNA Immobilization Efficiency)

The aminated oligo DNA was dissolved at 0.5 mg/mL concentration in “Aldehyde Spotting Solution” (product of GENEPAK Company) to prepare a DNA spotting solution. By a DNA microarray spotter (manufactured by Nichiryo Co., Ltd.), the DNA spotting solution was spotted on the individual substrates, followed by incubation at 37° C. for 30 minutes and then at 80° C. for 60 minutes. NaBH₄ (0.5 g) was dissolved in ethanol (13.3 mL) and PBS(−) (45 mL) to prepare a blocking solution. The substrates were immersed for 5 minutes in the blocking solution, washed with pure water, treated for 3 minutes in boiling water, immersed for 1 minute in ice-cold ethanol, and then dried in air.

The rhodamine-labeled oligo DNA was dissolved in 5×SSC solution, which contained 0.2% of SDS, to prepare a rhodamine-labeled oligo DNA solution. The rhodamine-labeled oligo DNA solution was subjected for 3 minutes to a boiling treatment, and then chilled with ice. The solution was then added dropwise (80 μL aliquots) onto the substrate on which the aminated oligo DNA had been immobilized. The substrate was covered with a cover glass, and then incubated under moisture retention at 60° C. for 18 hours. Subsequent to removal of the cover glass, the substrate was washed successively with 2×SSC which contained 0.5% of SDS, 0.5×SSC and pure water in this order, dried in air, and then provided for a comparison in the immobilized amount of DNA.

A comparison in the immobilized amount of DNA was conducted as will be described next. By commonly setting an exposure time and the like, pictures of fluorescent images of rhodamine were taken with a fluorescence microscope (manufactured by Olympus Corporation) while focusing on the individual spots. The pictures were read as image data by an image scanner, the image data were subjected to image processing to convert the intensity of the fluorescence into digital data, and the digital data were then compared as immobilized amounts of the aminated oligo DNA. Assuming that the average value of the individual spots in Example 1 was 100, immobilized amounts on the individual substrates were compared and intensity variations among the spots were also compared. The results are shown in Table 1.

(Comparison in Detection Intensity Upon Hybridization)

The aminated oligo DNA was dissolved at 0.5 mg/mL concentration in “Aldehyde Spotting Solution” (product of GENEPAK Limited) to prepare a DNA spotting solution. By a DNA microarray spotter (manufactured by Nichiryo Co., Ltd.), the DNA spotting solution was spotted on the individual substrates, followed by incubation at 37° C. for 30 minutes and then at 80° C. for 60 minutes. NaBH₄ (0.5 g) was dissolved in ethanol (13.3 mL) and PBS(−) (45 mL) to prepare a blocking solution. The substrates were immersed for 5 minutes in the blocking solution, washed with pure water, treated for 3 minutes in boiling water, immersed for 1 minute in ice-cold ethanol, and then dried in air.

The Cy3-labeled cDNA was dissolved in 5×SSC solution, which contained 0.2% of SDS, to prepare a Cy3-labeled cDNA solution. The Cy3-labeled cDNA solution was subjected for 3 minutes to a boiling treatment, and then chilled with ice. The solution was then added dropwise (80 μL aliquots) onto the substrate on which the aminated oligo DNA had been immobilized. The substrate was covered with a cover glass, and then incubated under moisture retention at 60° C. for 18 hours. Subsequent to removal of the cover glass, the substrate was washed successively with 2×SSC which contained 0.5% of SDS, 0.5×SSC and pure water in this order, dried in air, and then provided for a comparison in the immobilized amount of DNA.

A comparison in the immobilized amount of DNA was conducted as will be described next. By commonly setting an exposure time and the like, pictures of fluorescent images of rhodamine were taken with a fluorescence microscope (manufactured by Olympus Corporation) while focusing on the individual spots. The pictures were read as image data by an image scanner, the image data were subjected to image processing to convert the intensity of fluorescence into digital data, and the digital data were then compared as immobilized amounts of the hybridized cDNA. Assuming that the average value of the individual spots in Example 1 was 100, immobilized amounts on the individual substrates were compared and intensity variations among the spots were also compared. The results are shown in Table 2.

TABLE 1
Comparison in Immobilized Amount of Oligo DNA

| | Average | Maximum | Minimum | CV value (%) |
|---|---|---|---|---|
| Example 1 | 100 | 121 | 86 | 13.5 |
| Example 2 | 95 | 112 | 85 | 14.5 |
| Comp. Ex. 1 | 55 | 70 | 30 | 19.5 |
| Comp. Ex. 2 | 32 | 40 | 15 | 25.3 |

TABLE 2
Comparison in Hybridized Amount of cDNA

| | Average | Maximum | Minimum | CV value (%) |
|---|---|---|---|---|
| Example 1 | 100 | 115 | 85 | 12.1 |
| Example 2 | 98 | 110 | 82 | 15.1 |
| Comp. Ex. 1 | 34 | 57 | 21 | 22.5 |
| Comp. Ex. 2 | 21 | 41 | 12 | 28.2 |

As evident from Table 1 and Table 2, the substrate according to the present invention is high in the efficiency of immobilization, is not observed to undergo a reduction in immobilization ability during long-term storage, assures uniform immobilization of DNA in spots upon immobilization of DNA by DNA spotting, and also assures small variations in intensities detected at the spots in the detection of DNA after hybridization.

Further, it is unnecessary to add glutaraldehyde to a solution upon immobilization of DNA. It is, therefore, possible to control small a loss of DNA which takes place due to crosslinking of DNA strand fragments themselves.

### Example 3

Using the same saturated cyclic polyolefin resin as that employed in Example 1, slide-glass-shaped substrates were obtained by injection molding. To the surfaces of those molded products, a hydrophilization treatment was applied by low-temperature oxygen plasma treatment. Next, a solution with γ-aminopropytriethoxysilane and methyltriethoxysilane dissolved at 2:1 to 5% concentration in methanol was prepared as a treatment solution for the introduction of amino groups. After the molded products were immersed for 2 hours in the solution, the resulting substrates were taken out of the solution, allowed to stand in ultrapure water, taken out of the ultrapure water, and then dried. Glutaraldehyde was dissolved at 2% concentration in PBS(−) to prepare a glutaraldehyde solution. The substrates which had been subjected to the aminoalkylsilane treatment were immersed in the glutaraldehyde solution. After the substrates were allowed to stand there for 4 hours, they were taken out of the solution, immersed and washed in ultrapure water, and then dried.

Using the substrates obtained in Examples 1 and 3 and Comparative Example 1, a comparison in the immobilized amount of DNA and a comparison in detection intensity upon hybridization were each conducted in a similar manner as described above. The results are shown in Table 3 and Table 4. Incidentally, the values in Tables 3 and 4 are indicated by assuming that the data of Example 3 were 100.

TABLE 3
Comparison in Immobilized Amount of Oligo DNA

| | Average | Maximum | Minimum | CV value (%) |
|---|---|---|---|---|
| Example 3 | 100 | 115 | 88 | 11.8 |
| Example 1 | 85 | 95 | 70 | 14.5 |
| Comp. Ex. 1 | 50 | 65 | 30 | 19.5 |

TABLE 4
Comparison in Hybridized Amount of cDNA

| | Average | Maximum | Minimum | CV value (%) |
|---|---|---|---|---|
| Example 3 | 100 | 110 | 85 | 11.1 |
| Example 1 | 90 | 100 | 75 | 13.1 |
| Comp. Ex. 1 | 32 | 57 | 20 | 22.5 |

From Tables 3 and 4, it is appreciated that, when an alkylsilane is caused to exist in addition to an aminoalkylsilane on the surface of a plastic substrate, the immobilization efficiency and hybridization efficiency of DNA can be improved further.

### Referential Example 1

Using the hydrogenation product of the random copolymer between ethylene and dicyclopentadiene as a norbornene derivative, slide plates were injection molded to obtain molded products A. By generating high-frequency low-temperature plasma while feeding oxygen gas under reduced pressure, the molded products A were subjected to an oxidation treatment for 10 minutes. Immediately after the oxidation treatment, the molded products A were immersed for 10 seconds in pure water.

### Comparative Referential Example 1

By generating low-temperature radio-frequency plasma while feeding oxygen gas under reduced pressure, the molded products A of the referential example were subjected to an oxidation treatment for 10 minutes.

[1] Autofluorescence

The slide plates of Referential Example 1 and Comparative Referential Example 1 were observed under an inverted fluorescence microscope (excitation light wavelength: 532 nm, fluorescence wavelength: 560 nm). Fluorescence was captured by a CCD camera, and from image data, intensities of fluorescence were compared and evaluated.

Evaluation results are shown in Table 5. From the evaluation results, it has been recognized that background fluorescence can be reduced by introducing hydroxyl groups onto π-bonded carbon atoms in the saturated cyclic polyolefin resin.

[2] ESCA Analysis

Specimens cut out from the molded products of Referential Example 1 and Comparative Referential Example 1, respectively, were subjected to a surface analysis by ESCA (model: F1, manufactured by SURFACE SYSTEM, INC., “ESCALAB 220i-XL”, analysis area: 0.6 mm across, photoelectron escape angle: 90 deg., degree of vacuum: 1.5×10⁻⁶ Pa, X-ray source: AlK α radiation), and the percentages of carbon atoms, which existed in the molecular layers making up the surfaces of the substrates, as broken down depending on the state of bonding were compared and evaluated. Evaluation results are shown in Table 6. From the evaluation results, it has been recognized that, by applying the treatments of the present invention, hydroxyl groups increase and carbon atoms having π-bonds decrease.

TABLE 5

| | | Comp. Ref. |
|---|---|---|
| | Ref. Ex. 1 | Ex. 1 |
| Oxygen gas low-temperature plasma | Applied | Applied |
| treatment | | |
| Pure water immersion treatment | Applied | Not applied |
| Intensity of fluorescence (molded | 107 | 217 |
| resin product = 100) | | |

TABLE 6

| | Ref. Ex. 1 | Comp. Ref. Ex. 1 |
|---|---|---|
| —CH₂— | 70.4 | 69.7 |
| —C—OH | 15.9 | 10.1 |
| >C═O | 7.2 | 8 |
| 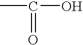 | 6.5 | 12.2 |

The substrates according to the present invention, which had been produced by using the substrates of Referential Example 1 and introducing an aminoalkylsilane with an aldehyde group bonded thereto in a similar manner as in Examples 1-3, were high in DNA immobilization efficiency and hybridization efficiency, and were reduced in fluorescence from the substrates themselves.

### Referential Example 2

Stabilizer wax (50 wt. %) was added to carbon black (50 wt. %), and pellets with the black pigment contained at high concentration were prepared. To the hydrogenation product (95 wt. %) of the random copolymer between ethylene and dicyclopentadiene, the above-mentioned pellets (5 wt. %) were added to prepare a black-colored, saturated cyclic polyolefin resin. Using that resin, slide plates of 1 mm thickness were injection-molded.

### Referential Example 3

To polystyrene (95 wt. %), the pellets (5 wt. %) described in Referential Example 2 were added to prepare black-colored polystyrene. Using that resin, slide plates of 1 mm thickness were injection-molded.

### Comparative Referential Example 2

Using the random copolymer between ethylene and dicyclopentadiene, slide plates of 1 mm thickness were injection-molded.

### Comparative Referential Example 3

Using polystyrene, slide plates of 1 mm thickness were injection-molded.

### Comparative Referential Example 4

A slide glass made of silica glass (“S1111”, product of Matsunami Glass Ind. Ltd.) was used. (The slide plates of Referential Examples 2 and 3 and Comparative Referential Examples 2, 3 and 4 were observed under an inverted florescence microscope (excitation light wavelength: 532 nm, fluorescence wavelength: 560 nm) without placing any sample or slide or anything thereon. Fluorescence was captured by a CCD camera, and from image data, intensities of fluorescence were compared and evaluated.

Evaluation results are shown in Table 7. From the evaluation results, it has been recognized that background fluorescence can be reduced by applying an opacification treatment with a pigment. In particular, the slide plate of Referential Example 2 has been recognized to be lower in background value than the silica glass of Comparative Referential Example 4.

TABLE 7

| | | Intensity of |
|---|---|---|
| | | fluorescence |
| | | (silica glass = 100) |
| Ref. Ex. 2 | Saturated cyclic polyolefin resin | 62 |
| | Colored in black | |
| Ref. Ex. 3 | Polystyrene colored in black | 105 |
| Comp. Ref. Ex. 2 | Saturated cyclic polyolefin | 262 |
| | Resin | |
| Comp. Ref. Ex. 3 | Polystyrene | 1,549 |
| Comp. Ref. Ex. 4 | Silica glass | 100 |

The substrates according to the present invention, which had been produced by using the resins of Referential Examples 2 and 3, especially the resin of Referential Example 3 and introducing an aminoalkylsilane with an aldehyde group bonded thereto in a similar manner as in Examples 1-4, were substrates having high DNA immobilization efficiency and hybridization efficiency and low fluorescence background.

### Referential Example 4

Using the same norbornene-based polyolefin resin as that employed in Example 1, a substrate shown in FIG. 1 was injection-molded. Its length and width were 76 mm and 26 mm, respectively, its sample-immobilizing section was 0.9 mm thick, and its outer edge portion was 1 mm thick. The surface roughness of the molded product was 0.002 to 0.003 μm, and neither a strain nor a warp was recognized throughout the substrate. The substrate was scanned using a microarray scanner, “ScanArray LITE” manufactured by Packard BioChip Technologies, Inc. As scanning conditions, the laser output and PMT sensitivity were set at 90% and 90%, respectively. As shown in the (upper) histogram of FIG. 5, the background fluorescence did not have much variations. It was, therefore, demonstrated that the surface of the substrate was free of deformation such as warp or strain.

### Referential Example 5

Using the same resin as that employed in Referential Example 4, a slide-glass-shaped substrate of 76×26×1 mm was injection-molded. The surface roughness of the molded product was 0.002 to 0.003 μm, and neither a strain nor a warp was recognized throughout the substrate. As a result of scanning under similar conditions as in Referential Example 4, variations were recognized in background fluorescence as shown in the (lower) histogram of FIG. 5. Those variations are attributed to a deformation of the surface of the substrate under a stress from a fixing jig.

The substrate according to the present invention, which had been produced by using the injection-molded product of Referential Example 4 and introducing an aminoalkylsilane with an aldehyde group bonded thereto in a similar manner as in Examples 1-3, was high in DNA immobilization efficiency and hybridization efficiency, and on its sample-immobilizing surface, was free of such a deformation that would otherwise occur under uneven force applied from the fixing jig.

### INDUSTRIAL APPLICABILITY

In the immobilization of DNA by its spotting, the microarray chip substrate according to the present invention is high in the immobilization efficiency of DNA, and the immobilized amount of DNA is even throughout the substrate. In hybridization, the substrate is also high in the hybridization efficiency of DNA, and upon detection, the detection intensities of spots do not vary much. The substrate is, therefore, suited as a DNA microarray substrate.

1. A plastic substrate for a microarray chip, characterized in that an aminoalkylsilane with an aldehyde group derived from glutaraldehyde and introduced in an amino group of said aminoalkylsilane exists on a surface of said plastic substrate.

2. The plastic substrate according to claim 1, wherein an aminoalkylsilane also exists on said surface of said plastic substrate.

3. The plastic substrate according to claim 1 or 2, wherein an alkylsilane also exists on said surface of said plastic substrate.

4. The plastic substrate according to claim 3, wherein said alkylsilane has a shorter molecular chain than said aminoalkylsilane.

5. The plastic substrate according to any one of claims 1-4, wherein said plastic is a saturated cyclic polyolefin.

6. The plastic substrate according to claim 5, wherein said saturated cyclic polyolefin resin comprises at least one of norbornene or a norbornene derivative as a monomer unit.

7. The plastic substrate according to any one of claims 1-6, wherein said plastic comprises a pigment and is opaque.

8. The plastic substrate according to claim 7, wherein said pigment is a black pigment.

9. The plastic substrate according to claim 7, wherein said pigment is a white pigment.

10. The plastic substrate according to any one of claims 7-9, wherein a content of said pigment is from 1 to 60 wt. %.

11. The plastic substrate according to any one of claims 1-10, wherein said substrate is provided on a back side thereof with a means for limiting an area of contact with a measuring system.

12. The plastic substrate according to claim 11, wherein said means for limiting said area of contact is a raised portion arranged on a part of a back side of a sample-fixing section of said substrate.

13. The plastic substrate according to claim 12, wherein said raised portion is an outer edge portion greater in thickness than said sample-fixing section.

14. A process for the production of a plastic substrate for a microarray chip, characterized in that an aminoalkylsilane with an aldehyde group derived from glutaraldehyde and introduced in an amino group of said aminoalkylsilane is caused to exist on a surface of said plastic substrate by steps which comprise:
(1) subjecting a starting plastic substrate to a surface oxidation treatment,
(2) bringing the thus-obtained plastic substrate into contact with a solution of said aminoalkylsilane, and
(3) bringing the resulting plastic substrate into contact with a solution of glutaraldehyde.

15. The process according to claim 14, wherein hydroxyl groups are introduced onto said surface of said plastic substrate by said surface oxidation treatment.

16. The process according to claim 14 or 15, wherein said surface oxidation treatment is low-temperature plasma treatment.

17. The process according to claim 16, wherein said low-temperature plasma treatment is conducted in oxygen or an oxygen-containing gas atmosphere.

18. The process according to any one of claims 15-17, wherein immediately after said oxidation treatment, hydroxyl groups are introduced onto carbon atoms which are in a radical state or are π-bonded.

19. The process according to claim 18, wherein a method for introducing hydroxyl groups onto carbon atoms, which are in a radical state or are π-bonded, immediately after said oxidation treatment comprises bringing said oxidation-treated into contact with water molecules.

20. The process according to any one of claims 14-19, wherein said plastic is a saturated cyclic polyolefin.

21. The process according to claim 20, wherein said saturated cyclic polyolefin resin comprises at least one of norbornene or a norbornene derivative as a monomer unit.

22. The process according to claim 20 or 21, wherein carbon atoms having π-bonds account for 15% or less of carbon atoms existing in a molecular layer which forms said surface of said substrate by introducing hydroxyl groups onto said π-bonded carbon atoms.

23. The process according to any one of claims 14-22, wherein said solution of said aminoalkylsilane further comprises an alkylsilane.

24. The process according to claim 22, wherein said alkylsilane has a shorter molecular chain than said aminoalkylsilane.

25. A method of use of a plastic substrate for a microarray chip characterized in that said method comprises dissolving in a solution DNA strands with amino groups introduced at ends thereof, bringing the resulting solution into contact with a substrate according to any one of claims 1-13, and having said amino groups of said DNA strands and aldehyde groups, which have been introduced onto said substrate, covalently bonded with each other to immobilize said DNA strands on said substrate.
